(19) United States
(12) Patent Application Publication
(10) Pub. No.: US 2010/0075420 A1
(43) Pub. Date: Mar. 25, 2010

(54) Novel Gene Delivery Vectors for Human Mesenchymal Stem Cells

(76) Inventors: Anita Saraf, Houston, TX (US); Michael C. Hacker, Leipzig (DE); Antonios G. Mikos, Houston, TX (US)

Correspondence Address: Baker Botts L.L.P, 910 Louisiana Street, One Shell Plaza, HOUSTON, TX, 77002, US

(21) Appl. No.: 12/563,181

(22) Filed: Sep. 21, 2009

(30) Foreign Application Priority Data
Mar. 14, 2008 (US) PCT/US2008/057014

(51) Int. Cl. C12N 15/00; C08L 5/00; C12N 15/63; C12N 5/00

(52) U.S. Cl. 435/440; 525/54.2; 435/320.1; 435/375

(57) ABSTRACT

Novel gene delivery vector compositions that interact with human mesenchymal stem cells are provided, as well as methods of synthesizing and using such compositions. Such compositions may comprise a plurality of hyaluronic acid hexamers covalently attached to a branched polyethylenimine. Such methods of synthesis may comprise providing a plurality of hyaluronic acid hexamers and a branched polyethylenimine, and allowing a hexamer of hyaluronic acid to covalently attach to a branched polyethylenimine to form a conjugate. Such methods of use may comprise providing a conjugate comprising a plurality of hyaluronic acid hexamers covalently attached to a branched polyethylenimine, and administering the conjugate to a cell.

### CROSS-REFERENCE TO RELATED APPLICATIONS

This application claims the benefit of U.S. Provisional Patent Application Ser. No. 60/896,105, filed Mar. 21, 2007, and PCT Patent Application No. PCT/US2008/057015, filed Mar. 14, 2008, the entire disclosures of which are incorporated herein by reference.

### STATEMENT OF GOVERNMENT INTEREST

The present invention was made with support under Grant Number EB 004963 awarded by the National Institutes of Health. The U.S. government has certain rights in the invention.

### BACKGROUND

The present disclosure generally relates to gene delivery vectors. More specifically, the present disclosure provides compositions and methods related to gene delivery vectors that interact with human mesenchymal stem cells.

In recent years, there has been heightened interest in mesenchymal stem cells for tissue engineering applications. In particular, researchers have focused much attention on marrow stomal cells that are derived from bone marrow. As used herein, the term “mesenchymal stem cells” includes these marrow stromal cells. Researchers have manipulated certain properties of mesenchymal stem cells with growth factors, gene delivery vectors, and mechanical stimuli. However, very few agents have combined these biological stimuli to promote mesenchymal stem cell differentiation.

The search for improved gene delivery vectors is an active field of research. As used herein, the term “gene delivery vector” refers to a molecule or molecules capable of interacting with DNA and facilitating the delivery and/or transfection of DNA into target cells. The term “transfection” refers to the introduction of exogenous DNA into a target cell. One substance that has shown promise as a non-viral gene delivery vector is branched polyethylenimine (“bPEI”). Researchers have described the dynamic behavior of bPEI at various pHs, salt concentrations, and temperatures. (Thomas and Klibanov, 2002). In previous studies, various ligands have been attached to bPEI with the objective of achieving specific targeting to the liver (Zanta et al. 1997) or the lungs (Grosse et al. 2004). However, bPEI is significantly toxic to certain cell types, including human mesenchymal stem cells (hMSCs). The toxicity of cationic polymers such as bPEI has been attributed to multiple structural characteristics of the cationic polymer itself. For example, the cytotoxicity of bPEI has previously been attributed to the spatial density of its cationic charges. (Godbey, Wu, and Mikos, 1999.) Additionally, Fischer et al. have reported that the toxicity of cationic polymers is dependent on the total number of primary amines as well as the overall density of amines, both in linear and three dimensional space. (Fischer et al. 2003).

Cellular uptake of DNA has been mainly attributed to either or both of two processes: cellular uptake during cell division, and receptor mediated endocytosis. One event that may trigger receptor mediated endocytosis is the binding of hyaluronic acid (“HA”) to CD44 receptors. During an extracellular matrix remodeling process, many cell types, including mesenchymal stem cells, engulf HA through CD44 receptors. This property makes HA an excellent candidate for use in gene delivery. However, the efficiency of a gene delivery vector is not based only upon its ability to interact with target cells; the sizes of the vector-DNA complexes formed are equally significant. Theoretical (Gao et al. 2005) and experimental studies (Rensen et al.) have shown that vector-DNA complexes are better transfection agents when they are charge-neutral and less than 50 nm in size. When gene delivery agents have a charge associated with them, the optimal size is pushed towards larger particles (˜100 nm) (Wagner et al., 1991).

In addition to its ability to trigger receptor mediated endocytosis by binding CD44 receptors, hyaluronic acid is extensively involved in tissue formation and reconstruction. The interaction between HA molecules and condensing mesenchymal cells is intricate and integral to tissue formation. Research groups including Knudson et al. (Hua et al. (1993); Knudson (2003); Knudson and Knudson (2004); Ohno et al. (2005)), Toole et al. (1972, 1997, 2001), and others (Chow et al (2006); Lisignoli et al. (2005); Seyfried et al. (2005)) have provided exhaustive scientific insight into HA's influence on modeling tissue development. In particular, it has been shown that HA, among other things, promotes cell condensation by anchoring cells via CD44 receptors and hyaluronan synthase, provides interactions for the cells with their extracellular matrix (ECM) via binding to ECM molecules, and influences cell behavior by promoting glycosylation, alternative splicing and clustering through intracellular signaling. Additionally, it has been shown that HA-based scaffolds are better promoters of osteochrondral healing (Solchaga et al., 2005) and vascularization than most other types of scaffolds. There is recent evidence that CD44-HA interactions promote cellular response to bone morphogenic proteins. (Peterson et al. 2004). HA hexamers similar to those used in the compositions and methods of the present invention have been shown to increase type II collagen expression, as well as upregulate some signaling molecules that are involved upstream (such as retinoic acid receptors) in chondrogenesis. (Knudson and Knudson, 2004). On the whole, the body of research is clear that HA interacts with various cell types during tissue formation and repair.

### SUMMARY

The present disclosure generally relates to gene delivery vectors. More specifically, the present disclosure provides compositions and methods related to gene delivery vectors that interact with human mesenchymal stem cells.

In certain embodiments, the present disclosure provides a composition comprising a plurality of hyaluronic acid hexamers covalently attached to a branched polyethylenimine.

In certain embodiments, the present disclosure provides a method comprising providing a plurality of hyaluronic acid hexamers and a branched polyethylenimine, and allowing a hexamer of hyaluronic acid to covalently attach to a branched polyethylenimine to form a conjugate.

In certain embodiments, the present disclosure provides a method comprising providing a conjugate comprising a plurality of hyaluronic acid hexamers covalently attached to a branched polyethylenimine, and administering the conjugate to a cell.

### DRAWINGS

Some specific example embodiments of the disclosure may be understood by referring, in part, to the following description and the accompanying drawings.

FIG. 1 shows a schematic of a possible reductive amination reaction between branched polyethyleneimine (bPEI) and hyaluronic acid (HA) hexamers. The structure of bPEI has been simplified for brevity. All structures are depicted in the undissociated state. The carbon atoms of the structural subunits of HA, N-acetylglucosamine (C1-C6) and glucuronate (C1′-C6′), are numbered for reference. bPEI represented in the products has two different forms of primary amines, those that participate in the chemical bond with HA and those that do not.

FIG. 2(A) shows the ¹H NMR spectra of bPEI-HA and HA.

FIG. 2(B) shows DEPT-135 ¹³C NMR spectra of bPEI-HA and HA.

FIG. 3 shows the product of Fluorescence Assisted Carbohydrate Electrophoresis. Lanes 1-8 are labeled with the samples loaded with (2,4,6) or without treatment (3,5,7) with Chondroitinase ACII.

FIG. 4 shows the results of agarose electrophoresis of bPEI-HA/DNA complexes assembled at a cation:anion ratio (“C:A ratio”) of 7.5:1 and 2 μg of DNA. bPEI-HA was equilibrated at various salt concentrations indicated, after which DNA was added and the solution was allowed to stand for 1 hr. The samples were loaded in the agorose gel and run at 70 V for 1.5 hr. As the salt concentration increases the amount of DNA visible in the gels decreases. (The colors of the image of the gel obtained have been inverted for aesthetic reasons).

FIG. 5 shows the dynamic light scattering distribution for bPEI-HA/DNA complexes assembled at C:A ratios of 7.5:1 at (A) 25° C. and (B) 37° C. Complexes tend to have a leftward shift when salt concentration is increased. However, with increase in temperature, the respective hydrodynamic radii for the corresponding NaCl solutions move to the right.

FIG. 6(A) shows the results of representative live dead assays performed at 24 hours and 72 hours after exposure to unconjugated bPEI, bPEI-HA conjugates, and bPEI-HA/DNA complexes. The groups are defined as follows: Group 1: 1 μg DNA, 1 μg bPEI or 3 μg bPEI-HA, (computes to an N:P and C:A ratio of 7.5:1 respectively) Group 2: 1 μg DNA, 1.8 μg bPEI or 5 μg bPEI-HA (computes to a C:A ratio of 13.5:1), Group 3: 2.56 μg DNA and 4.5 μg bPEI or 12.3 μg bPEI-HA. Error bars indicate 1 standard deviation. Replication per sample was n=4. Asterisks (*) indicate significant difference between bPEI-HA and bPEI and bPEI-HA/DNA and bPEI evaluated with P-values <0.05.

FIG. 6(B) shows the images of cells treated with bPEI-HA conjugates (left) and an equivalent amount of unconjugated bPEI (right) after 8 hours of exposure to the respective gene delivery vectors. The uptake of red dye by the cells on the left as well as clumping signifies apoptosis of cells.

FIG. 7(A) shows the mean fluorescence of hMSCs at 48 hours and 72 hours post transfection. Groups compared include “control” (DNA only); with a cation:anion ratio of 7.5:1, complexes assembled at 25° C. in 150 mM NaC; with C:A ratio of 7.5:1, complexes assembled at 37° C. at 150 mM NaCl solution; with C:A ratio 7.5:1, complexes assembled at 25° C. and 0 mM NaCl solution. Error bars represent one standard deviation at n=4 and asterisks (*) represent statistical difference between the groups indicated with P-values <0.05.

FIG. 7(B) shows representative images of fluorescence of hMSCs treated with (a) DNA alone, (b) complexes assembled at 25° C. in 150 mM NaCl, (c) at 37° C. and 150 mM NaCl and (d) 500 mM NaCl and 25° C. bPEI-HA/DNA vectors assembled at C:A ratios of 7.5:1 and 4 μg of DNA/50,000 cells.

FIG. 8 shows a schematic of a proposed hypothesis explaining the behavior of bPEI-HA conjugates and bPEI-HA/DNA complexes in response to NaCl and/or temperature. NaCl may screen the ionic charges of bPEI-HA conjugates, causing the molecule to unfold (A). However, with bPEI-HA/DNA complexes, the addition of salt may cause separation of bPEI-HA/DNA complexes from each other, whereas adding heat may cause them to slightly unfold their conformation (B). Double headed arrows represent hypothesized interactions between the negative carboxyl groups of (HA)₆ and positive amine groups of bPEI.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to gene delivery vectors. More specifically, the present disclosure provides compositions and methods related to the use of a bPEI-HA conjugate as a gene delivery vector. One aspect of the invention provides compositions that comprise a bPEI-HA conjugate. Another aspect of the invention provides methods of synthesizing bPEI-HA conjugates. Still another aspect provides methods of using a bPEI-HA conjugate as a gene delivery vector, including methods which improve the efficiency of transfection into target cells.

One aspect of the present provides compositions comprising a bPEI-HA conjugate. In some embodiments, bPEI-HA conjugates may contain a plurality of HA hexamers which are bonded to a plurality of side branches on a bPEI molecule. In certain embodiments, the bonds between the HA hexamers and the bPEI side chains may be covalent bonds. It is believed that the covalent attachment of negatively charged HA hexamers to cationic bPEI molecules, in some cases, is responsible for certain differences in the way bPEI-HA conjugates respond to temperature and salt (e.g. NaCl) concentration as compared to unconjugated bPEI. Based on theoretical calculations, it is believed that certain bPEI-HA conjugates may have an overall positive charge. In certain embodiments, the bPEI-HA conjugates of the present invention have a degree of substitution at the primary amine groups of bPEI with HA in the range of from about 12% to about 14%. At some pH levels, e.g., physiological pH, the conformation of certain bPEI-HA conjugates may be controlled by both hydrogen bonds as well as ionic bonds. Proton and ¹³C NMR spectrometry and/or Fluorescence Assisted Carbohydrate Electrophoresis (“FACE”) may be used to confirm and/or characterize the structure of bPEI-HA conjugates.

Among the many potential advantages of the present invention, one advantage may be that bPEI-HA conjugates may demonstrate reduced cytotoxicity compared to unconjugated bPEI. Another potential advantage is that hMSCs and chondrocytes may interact with bPEI-HA conjugates more readily and/or in different ways than similar cells interact with unconjugated bPEI. These interactions may include, but are not limited to, the uptake of the bPEI-HA conjugate through endocytosis via matrix receptors (e.g., CD44 receptors).

The bPEI contained in bPEI-HA conjugates may be obtained or synthesized from any source. In certain embodiments, bPEI that is suitable for use in the compositions and methods of the present invention may obtained from Sigma-Aldrich of St. Louis, Mo. In certain embodiments, suitable HA hexamers have a molecular weight of approximately 2.3 kD and may be obtained from Genzyme Corp. of Cambridge, Mass.

According to some embodiments, the bPEI-HA conjugates of the present invention may have limited solubility in water, and may form visible clumps or aggregates when placed in pure water. The same bPEI-HA conjugates may be soluble in salt solutions having a concentration of at least 150 mM. By way of explanation and not of limitation, it is believed that salt ions in the solution may shield some of the inter-molecular ionic interactions that cause polymeric bPEI-HA conjugates to aggregate, e.g., to form visible clumps. One possible source of intermolecular ionic interactions is the attraction between the negative carboxyl groups of (HA)₆ and the positive amine groups of bPEI on neighboring bPEI-HA conjugates.

In some embodiments of present invention, the bPEI-HA conjugates are complexed with at least one DNA molecule or a portion of a DNA molecule, resulting in a composition referred to herein as a “bPEI-HA/DNA complex.” DNA suitable for use in the present in the invention is any DNA comprising a plasmid of interest. As used herein, the term “plasmid of interest” encompasses any plasmid that may desirably be transfected into target cells. In some embodiments, bPEI-HA/DNA complexes may be formed by allowing bPEI-HA conjugates to interact with DNA. By way of explanation and not of limitation, it is believed that negatively charged DNA interacts with positive charges on the bPEI backbone of the bPEI-HA conjugates to form a bPEI-HA/DNA complex.

In some embodiments in which a bPEI-HA conjugate interacts with DNA to form a bPEI-HA/DNA complex, the bPEI-HA conjugate is dissolved in an aqueous salt solution before complexation. Preferably, the concentration of the salt solution is at least 150 mM. In certain embodiments, the concentration of the salt solution in which bPEI-HA conjugates are dissolved is related to how much DNA the bPEI-HA conjugates are able to bind. For example, the concentration of salt solution that allows complete interaction between bPEI-HA conjugates and DNA may be determined by gel electrophoresis of solutions of varying salt concentration which also contain bPEI-HA conjugates and DNA. Visible streaks of DNA in lanes corresponding with solutions of relatively low salt concentrations may indicate that not all of the introduced DNA was bound to the bPEI-HA conjugates. However, lanes corresponding to higher NaCl concentrations may not show any residual DNA streaks, which suggests that the all of the introduced DNA was bound to the bPEI-HA conjugates, e.g. the DNA was completely “packed” inside the conjugates. By way of explanation, it is thought that in addition to salt ions causing bPEI-HA conjugates to dissolve in solution by shielding attractive inter-molecular forces, salt ions may also shield attractive intra-molecular forces, causing the bPEI-HA conjugates to unfold. In an unfolded state, even more of the positives charges of the bPEI backbone are available for binding DNA.

According to some embodiments in which bPEI-HA/DNA complexes are present in salt solution, the size of the bPEI-HA/DNA complexes is inversely related to the concentration of salt. For example, the hydrodynamic radii of bPEI-HA/DNA complexes may become smaller in increasingly concentrated salt solutions, e.g., a distribution curve of hydrodynamic radii obtained through dynamic light scattering studies may shift to the left with increasing salt concentration. Furthermore, these distribution curves may provide evidence that aggregates of bPEI-HA/DNA complexes decrease in size, e.g., break into smaller pieces, in response to increasing salt concentrations. This effect may be observed through bimodal distribution curves which show increased intensity at the lower mode and an approximately simultaneous increase in the intensity of the higher mode as salt concentration increases. Although distribution curves obtained from dynamic light scattering studies may be subject to Rayleigh scattering (the intensity of scattering is proportional to the sixth power of the radius of the particles, hence, larger particles scatter at much greater intensity than smaller particles), the intensity does not represent the percentage of the complexes at the corresponding hydrodynamic radius.

In certain embodiments, aggregates of the bPEI-HA/DNA complexes of the present invention may not disperse in response to heat. For example, at similar salt concentrations, the hydrodynamic radii of the bPEI-HA/DNA complexes may be longer at 37° C. than at 25° C. Although the reasons that bPEI-HA/DNA complexes increase in size with increasing temperature are not fully understood, it is thought the addition of heat causes individual bPEI-HA/DNA complexes to increase in thermal energy, thus causing their individual hydrodynamic radii to lengthen. Static light scattering studies have shown that bPEI-HA/DNA complexes at 25° C. and 37° C. have similar molecular weights at a given salt concentration. Another possible explanation for the observed increase in the size of bPEI-HA/DNA complexes is that at temperatures approaching 37° C., the complexes may undergo a conformational change, orienting the attractive forces between them so that even larger aggregates of complexes are formed. Yet another explanation may be that the addition of heat causes the bonds between the DNA and the bPEI-HA conjugate to loosen.

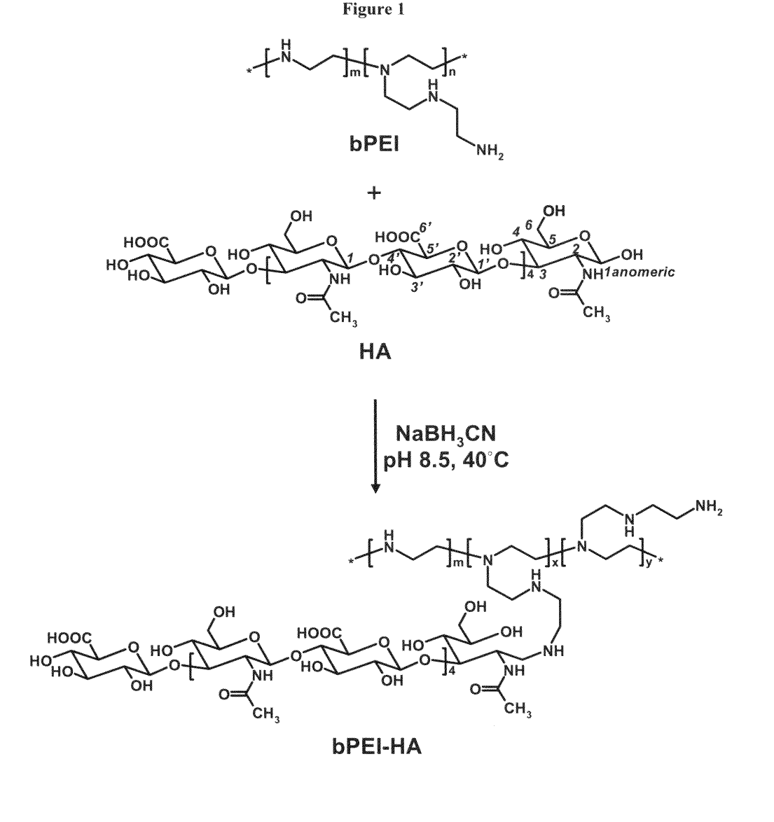

Another aspect of the present invention comprises methods of synthesizing bPEI-HA conjugates. According to one embodiment, bPEI-HA conjugates may be synthesized through a reductive amination reaction. One example of a reductive animation reaction that may represent a suitable method of synthesis is illustrated in FIG. 1. In preferred embodiments, the reaction may be carried out in an aqueous buffer. One preferred source for suitable chemicals for use in synthesis methods is Sigma Aldrich of St. Louis, Mo.

By way of explanation and not of limitation, in some embodiments in which bPEI-HA conjugate is synthesized, an imide intermediate may form at the anomeric C1 of HA. The intermediate is then reduced to a secondary amine by sodium cyanoborohydride. The bPEI-HA conjugates may be purified by ultrafiltration. An optional additional step in preparing the bPEI-HA conjugates is the lyophilization of the conjugates, which may, inter alia, convert the bPEI-HA conjugates into a form suitable for long-term storage. According to some embodiments, products obtained from one of the synthesis methods of the present invention may be characterized using ¹H and ¹³C nuclear magnetic resonance (“NMR”) spectroscopy.

Still another aspect of the present invention provides methods of using a bPEI-HA conjugate as a gene delivery vector, e.g., to transfect target cells with exogenous DNA. According to certain embodiments, a bPEI-HA conjugate is allowed to interact with DNA so that a bPEI-HA/DNA complex is formed. The bPEI-HA/DNA complex interacts with a target cell so that at least a portion of the DNA in the BPEI-HA/DNA complex is transfected into the target cell. In preferred embodiments, the target cells comprise hMSCs.

One possible advantage of using bPEI-HA conjugates as gene delivery vectors is that the conjugates may be less toxic to cells than unconjugated bPEI. For example, when exposed to unconjugated bPEI, less than 10% of hMSCs may survive an exposure period of 96 hours. However, more than 95% of hMSCs may survive similar exposure to bPEI-HA conjugates. Additionally, the reduced cytotoxicity of bPEI-HA conjugates may persist even when the bPEI-HA conjugates are complexed are with different concentrations of DNA. It is believed that reduced cytotoxicity of bPEI-HA conjugates compared to unconjugated bPEI is observed because covalent bonds between HA and bPEI mitigate some of the bPEI's cationic charge density by introducing anionic groups, and/or by reducing the total number of primary amines (e.g., when bPEI is complexed to HA through a reductive amination reaction, approximately 12% of the bPEI's primary amines may be changed to secondary amines). In some embodiments, bPEI-HA conjugates may be substantially non-toxic to hMSCs even with amine concentrations higher than the amine concentration of unconjugated bPEI by almost one order of magnitude (e.g. 1.2×10⁻⁶ M_{bPEI-HA} versus 2.3×10⁻⁷ M_{bPEI}).

In some embodiments, the success of transfecting target cells with bPEI-HA/DNA may depend on a number of factors, including the size of the bPEI-HA/DNA complexes and the process by which the bPEI-HA/DNA complexes are formed (e.g., how strongly the DNA binds to the bPEI-HA conjugates, and how much or how efficiently the DNA binds to the conjugates).

Some embodiments of the present invention relate to tailoring the synthesis or environment of bPEI-HA conjugates and/or bPEI-HA/DNA complexes to increase the efficiency with which bPEI-HA/DNA complexes are transfected into target cells. By way of explanation, factors which may be directly related to transfection efficiency include the strength of DNA binding and the density of DNA packing in bPEI-HA/DNA complexes and the size of the aggregates of bPEI-HA/DNA complexes that interact with the target cells. These properties may be modulated by varying the salt concentration and/or temperature of the solutions containing the complexes.

For example, in some embodiments of the present invention, the transfection of target cells (e.g., hMSCs) with bPEI-HA/DNA complexes may be increased by increasing the concentration of salt in the solution in which the bPEI-HA/DNA complexes form and/or in which the complexes contact hMSCs. In certain embodiments, transfection efficiency of bPEI-HA/DNA complexes assembled in 500 mM of salt may be statistically better than the transfection efficiency of complexes assembled in 150 mM salt. It is thought that the increase in salt concentration may (1) allow the bPEI-HA conjugates to better unfold during its complexation with DNA, thus allowing for better binding and packing of the DNA with the conjugates; (2) decrease the size of aggregates of bPEI-HA/DNA complexes, which increases the convenience of endocytosis; and (3) increase interaction between HA and the CD44 receptors on the hMSCs due to neutralization of the ionic repulsion between them, as both HA and CD44 are negatively charged.

In some embodiments of the present invention, the transfection of hMSCs with bPEI-HA/DNA complexes may be increased by decreasing the temperature at which bPEI-HA conjugates interact with DNA to form complexes, i.e. the temperature during assembly of the bPEI-HA/DNA complexes. Although the inverse correlation between transfection efficiency and assembly temperature is not fully understood, it is believed that increased temperatures cause the hydrodynamic radius of the bPEI-HA/DNA complexes to increase (e.g., allowing the bPEI-HA/DNA complexes to unfold). In certain example embodiments, bPEI/DNA complexes assembled at 37° C. may have lower transfection efficiency than the samples assembled at 25° C., although the difference may not be statistically significant.

To facilitate a better understanding of the present invention, the following examples of specific embodiments are given. In no way should the following examples be read to limit or define the entire scope of the invention.

### EXAMPLES

### Example 1

### Synthesis of bPEI-HA Conjugates

Chemicals used for synthesis of bPEI conjugates (including sodium borate, sodium cyanoborohydrate and bPEI) were obtained from Sigma-Aldrich of St. Louis, Mo. Hyaluronic acid hexamers (Mw=2.3 kDa) were obtained from Genzyme Corp of Cambridge, Mass. bPEI-HA conjugate was synthesized by the process of reductive amination. 250 mg of bPEI and 500 mg of HA were added to a three-neck round bottom flask in the presence of 0.1M Sodium Borate buffer (pH 8.5). Sodium cyanoborohydrate (0.2 mg) was added as a reducing agent at the beginning of the reaction and the mixture was heated to 40° C. Constant stirring was applied. After 30 hours, an additional 0.15 mg of sodium cyanoborohydrate was added into the reaction. The reaction was maintained for 48 hours, after which it was cooled. The products were dialyzed against 0.02 M of sodium borate buffer in a VivaSpin centrifuge tube (MWCO 30 kDa) (Sartorius Corp., Edgewood, N.Y.). The dialysate was gradually changed to pure water. The dialyzed products were then lyophilized and the dried powder thus obtained was retained for use for future characterization and experimentation.

### Example 2

### Characterization of bPEI-HA Conjugates by ¹H and ¹³C Nuclear Magnetic Resonance Spectroscopy

Samples of bPEI, HA, and bPEI-HA conjugates (prepared and powdered as described in Example 1) were prepared by dissolving the materials in D₂O at 37° C. on a shaker table. NMR spectra were recorded at ambient temperature and processed using the MestRe-C software (Mestrelab Research S.L., Spain). Proton NMR spectra were acquired on a 400 MHz spectrometer (Bruker Avance 400, Zurich, Switzerland). A Bruker Avance500 spectrometer was used to acquire DEPT-135 ¹³C NMR spectra. To improve signal-to-noise, line broadening of 2 Hz and 10 Hz was used to process the FID of the proton and carbon spectra, respectively. The chemical shifts in the spectra obtained were expressed as parts per million using HDO (δ=4.79 ppm) as an internal reference.

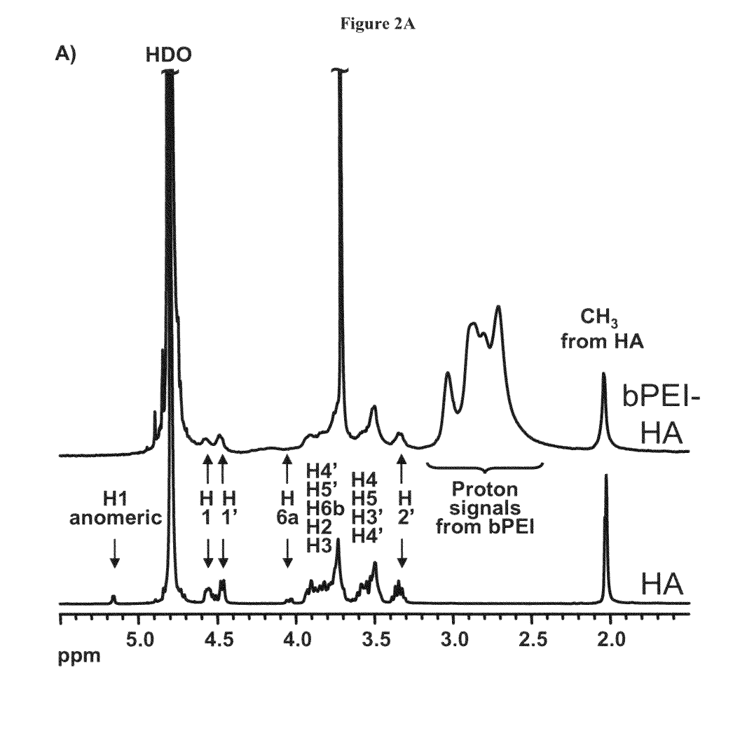

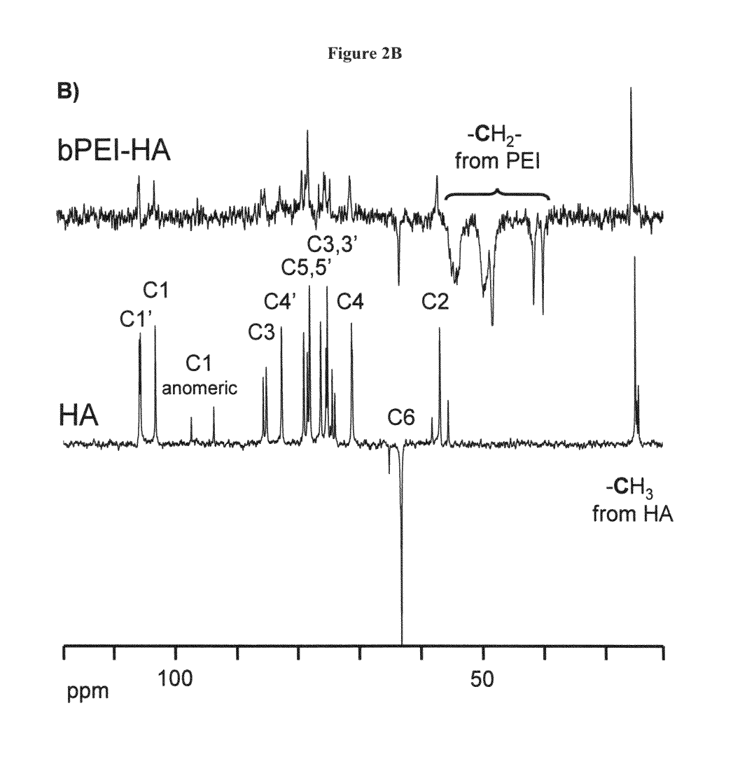

As shown in FIG. 2(A), the signal of the proton at the anomeric carbon C1 of HA was found at δ˜5.15 ppm in the HA spectrum and did not appear in the spectrum recorded for the purified bPEI-HA conjugate. Signals corresponding to all further functional groups of HA and bPEI were found in the NMR spectrum of the conjugate: δ˜2.0 ppm (H of —NCOCH3 from HA); δ 2.5-3.2 ppm (H of N—CH2-CH2-N from PEI); δ˜3.35 ppm (H of C2′ from HA); δ 3.4-3.7 ppm (H of C4, C5, C3′, C4′ from HA); δ 3.7-4.0 ppm (H of C2, C3, C6a, C4′, C5′ from HA); δ˜4.15 ppm (H of C6a from HA); δ˜4.45 ppm (H of C1′ from HA); δ˜4.55 ppm (H of C1 from HA).

The DEPT-135 ¹³C NMR spectrum of bPEI-HA shown in FIG. 2b further confirmed the presence of PEI and HA in the conjugate. The peaks of the functional groups can be assigned as follows: 24, 25: δ˜25 ppm (NCOCH3 from HA); δ 40-55 ppm (methylene N—CH2-CH2-N from PEI); δ˜57 ppm (methine C2 from HA); δ˜63 ppm (methine C6 from HA); δ˜71 ppm (methine C4 from HA); δ 74-76 ppm (methine C3, C3′ from HA); δ 78-80 ppm (methine C5, C5′ from HA); δ˜82 ppm (methine C4′ from HA); δ˜88 ppm (methine C3 from HA); δ˜103 ppm (methine C1 from HA); δ˜106 ppm (methine C1′ from HA). The C1 signal of α- and β-anomer was only found in the spectrum of HA at δ˜94 ppm and δ˜98 ppm. Due to a low signal-noise ratio in the bPEI-HA spectrum the absence of the anomeric C1 signal could not be confirmed as clearly as for the ¹H spectrum.

### Example 3

### Characterization of bPEI-HA Through Fluorescence Assisted Carbohydrate Electrophoresis

To quantify the amount of HA attached to bPEI in bPEI-HA conjugates, Fluorescence Assisted Carbohydrate Electrophoresis (“FACE”) with 2-aminoacridone (AMAC) was performed. 10 mg of HA and bPEI-HA conjugate were dissolved in samples of 150 mM NaCl solution and the pH was adjusted to 5.5 with the addition of phenol red. 5 μl of the samples were mixed with 80 μl of 0.1 M ammonium acetate and treated with 15 μl of chondroitinase ACII overnight at 37° C. The following day the samples were lyophilized and reconstituted with 0.1 M AMAC solution (85% of total volume) and in glacial acetic acid-DMSO (3:17, v/v, 15% of total volume) and freshly prepared solution sodium cyanoborohydride solution (3×10-5 M/sample). Then the mixtures were centrifuged for 5 min at 13,000 rpm. To allow derivatization with AMAC, the solution was incubated at 37° C. overnight. Fluorescent standards were prepared by serially diluting a known quantity of maltotriose and performing the same fluorescent labeling. After the derivatization procedure, samples were mixed with 20 μl of glycerol and covered in foil for FACE analysis.

For the preparation of polyacrylamide gels, two sets of solutions were prepared: (a) resolving gel solution (final concentration 20% acrylamide/bis-acrylamide (acryl/bis) (37.5:1), 2.5% glycerol and 44.8 mM tris acetate (pH 7.0) to a total volume of 5 ml); and (b) stacking gel solution (final concentration 8% acryl/bis (37.5:1), 44.8 mM tris acetate (pH 7.0), 2.5% glycerol and PEG (MW 8000, 4.4% w/v) for a total volume of 5 ml). The solutions were made fresh each time. Directly before adding to the plates, 28 μl of 10% ammonium persulfate and 7.5 μl TEMED were added. The solutions were mixed rapidly and then placed between glass plates, such that the stacking gel was added to the top and the resolving gel was at the bottom. An 8-10 well comb was inserted before the stacking gel polymerized.

8 μl of each sample (the experimental samples of interest together with the maltotriose samples for quantification) was loaded in each well. Electrophoresis was performed at 500 V until satisfactory resolution of the bands was obtained (60-75 minutes). The gels were illuminated with UV light and digitally imaged using a Kodak Gel Logic 100 imaging system and Kodak 1D software (Kodak, Rochester, N.Y., version 3.6.0). Quantitative analysis was performed with densitometery on Gel Pro Analyzer software (version 4.5.0, Media Cybernetics, Silver Spring, Md.).

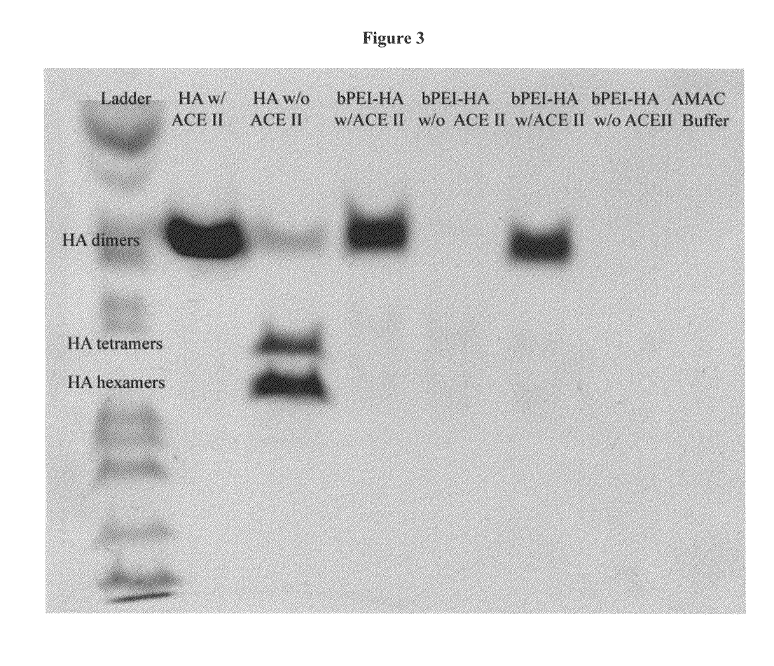

A sample gel containing HA and bPEI-HA conjugates, both treated and untreated with Chondroitinase ACII, is shown in FIG. 3. As can be seen, the undigested HA sample has a mixture of dimers, tetramers, and hexamers mixed within a sample obtained from the manufacturer. When treated with Chondroitinase ACII, which has hyaluronidase activity, these mutimers of HA all reduced to dimers. Lane 5 and lane 7, which contained bPEI-HA conjugates untreated with Chondroitinase ACII, contained no unattached HA, i.e., there were no non-covalent associations between bPEI and HA. When the same samples were treated with Chondroitinase ACII however (i.e., in lanes 4 and 6), HA dimers were apparent, which suggests that HA was covalently attached to bPEI. The amount of HA attached to bPEI was calculated using densitometry studies on the bands of HA on the gels. The relative ratios of dimers, tetramers and hexamers within the HA used were determined from lane 3. These ratios were then applied to the HA band in lanes 4 and 6, while standards were used to quantify the total HA dimers present in lanes 4 and 6.

Two more gels were run with similar solutions to determine the average amount of HA attached to bPEI. Calculations showed that, taking into account the various “mers” of HA present within the reactant HA, 12%-14% of the primary amine groups of bPEI were substituted with HA via reductive amination. Assuming that one dimer of HA carries one negative charge, whereas one “monomer” of PEI carries three positive charges contributed by primary and secondary amine groups (which are positively charged at physiological pH), the net charge on a molecule of bPEI-HA conjugate was determined.

### Example 4

### Determining the Efficiency of bPEI-HA/DNA Complexation Using Gel Electrophoresis

bPEI-HA/DNA complexes at cation:anion ratios (“C:A ratios”) of 7.5:1 and 13.5:1 were assembled in NaCl solutions of molarities ranging from 150 mM to 700 mM. (The total number of cations at physiological pH was determined by the total number of primary and secondary amines in bPEI-HA conjugates, the total number of anions was determined as the total carboxyl acid moieties (6 per hexamer of HA) contributed by HA). The samples were vortexed to allow complete mixing and then centrifuged at 10,000 rpm for 1 min. The samples were then allowed to stand for 1 hour before loading them on 1% agarose gels. For synthesis of the agarose gels, 0.5 g of agarose was added to 50 ml of 0.5× Tris-borate-EDTA (TBE) buffer. Dissolution was facilitated with heat, after which 1 μl of ethidium bromide was added to the solution. The gel was poured into a tank and after it was allowed to set, the gel was placed in 0.5×TBE solution. The samples were loaded in the wells with the addition of the loading buffer. The gel was run for 1.5 hr at 80 V after which the displacement of the bPEI-HA/DNA samples was monitored under a UV light.

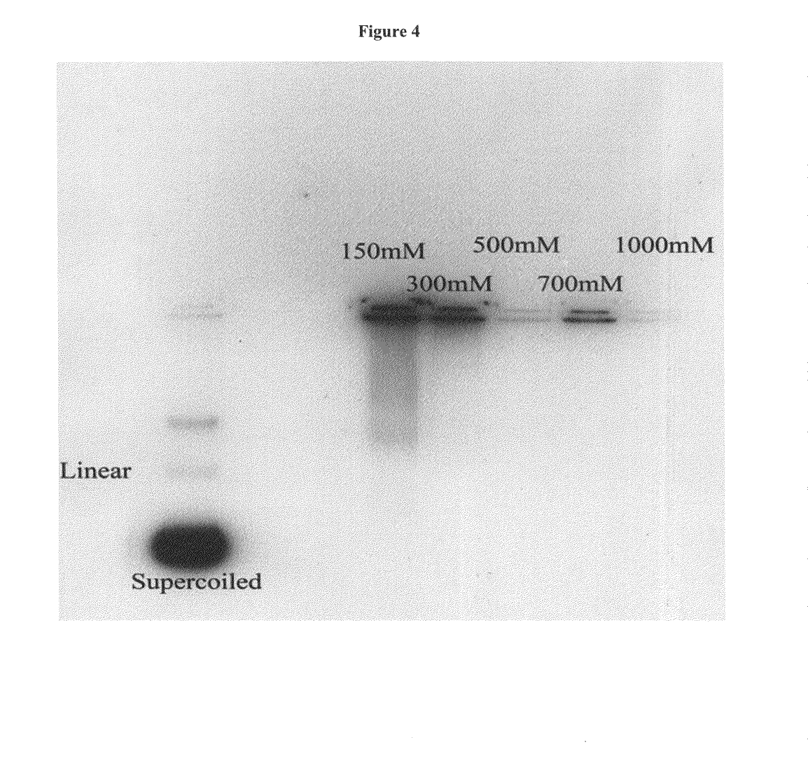

As can be seen from FIG. 4, the density of unbound DNA detected on the agarose gels decreased at increasing salt concentrations. At NaCl solution concentrations equal to or greater than 500 mM, no unbound DNA was observed in the agarose gel lanes. However, at NaCl concentrations of 150 mM and 300 mM, visible streaks were present in the agarose gel lanes. Also, no visible aggregates were seen in the solutions, suggesting that the complexes were completely dissolved.

### Example 5

### Characterization of bPEI-HA Conjugates Using Dynamic Light Scattering Techniques

To determine whether NaCl concentration affects the hydrodynamic radius (Rh value) of bPEI-HA conjugates, dynamic light scattering (“DLS”) studies were used to measure the hydrodynamic radius of bPEI-HA conjugates in NaCl solutions of varying concentration. A hydrodynamic radius obtained from DLS measurements represents the radius of a hypothetical sphere having the same diffusion constant as that of the polymeric substance being measured. Since the polymer is present in the form of a random coil, the Rh values obtained do not correspond to the actual values of aggregate sizes in solution. The Rh values are presented only to give qualitative information about aggregate behavior as a function of salt concentration and parameter.

To create stock solutions, bPEI-HA conjugates were dissolved in 150 mM, 300 mM, 500 mM, 700 mM, 1000 mM NaCl solution to make a 0.1 M bPEI-HA solution. The pH was adjusted to 7.4 using 0.5 N HCl in 150 mM-1000 mM NaCl solutions and filtered by passing through a 0.2 μm filter (Whatman, N.J.). Part of the above solutions were mixed with DNA (plasmid-CMV-BMP2) at a cation:anion ratio of 7.5:1. For DLS and Static Light Scattering (“SLS”) experiments, the above solutions were made fresh as 250 μl aliquots at 25° C., filtered through a 0.2 μm filter.

Hydrodynamic radii DLS results were obtained on a 90PLUS Particle Size Analyser (Brookhaven Instruments) operating at 659 nm wavelength laser. Samples of the stock solutions were allowed to equilibrate at 25° C. after introducing them into the cuvette for 5 min after which readings were obtained. The temperature was then raised to 37° C. and allowed to equilibrate with the solution for 10 min. The cumulant method was used to derive information about the Rh distribution in the form of the polydispersity index, and a Laplace inverse program called Non-Negative Least-Squares (NNLS) was used to determine the intensity weighted aggregate particle size in the form of hydrodynamic radius. The dust-cut off was set at 1000 nm and values higher than 1000 nm were not accounted for during processing of data.

As illustrated in Table 1, dynamic light scattering studies on bPEI-HA conjugates showed that the hydrodynamic radius of the conjugates increased with increasing salt concentration. In 150 mM NaCl solutions at physiological pH, bPEI-HA showed peak hydrodynamic radius at 1.78 nm. The distribution of hydrodynamic radii of bPEI-HA shifted significantly to higher hydrodynamic radii as higher molarity NaCl solutions were used. As can be seen in Table 1, the peak intensity occurred at 1.80 nm for the vector dissolved in 150 mM of NaCl, 10.6 nm for 500 mM of NaCl, and 24.4 nm at 700 mM NaCl.

TABLE 1
Rh values of bPEI-HA Conjugates
in Varying Concentrations of NaCl

| NaCl Concentration | 150 mM | 500 mM | 700 mM |
|---|---|---|---|
| Range of Rh (nm) | 1.0-3.2 | 4.0-27.0 | 14.3-71.0 |
| Mean Rh (nm) | 1.78 | 10.6 | 24.4 |

### Example 6

### Characterization of bPEI-HA/DNA Complexes Using Dynamic Light Scattering Techniques

To determine whether NaCl concentration affects the hydrodynamic radius of bPEI-HA/DNA complexes, dynamic light scattering studies were performed on bPEI-HA/DNA complexes. The procedures used substantially conformed with the procedures used in Example 5. Readings were taken at both 25° C. and 37° C. with bPEI-HA/DNA complexes at NaCl concentrations of 150 mM, 300 mM, 500 mM, and 700 mM.

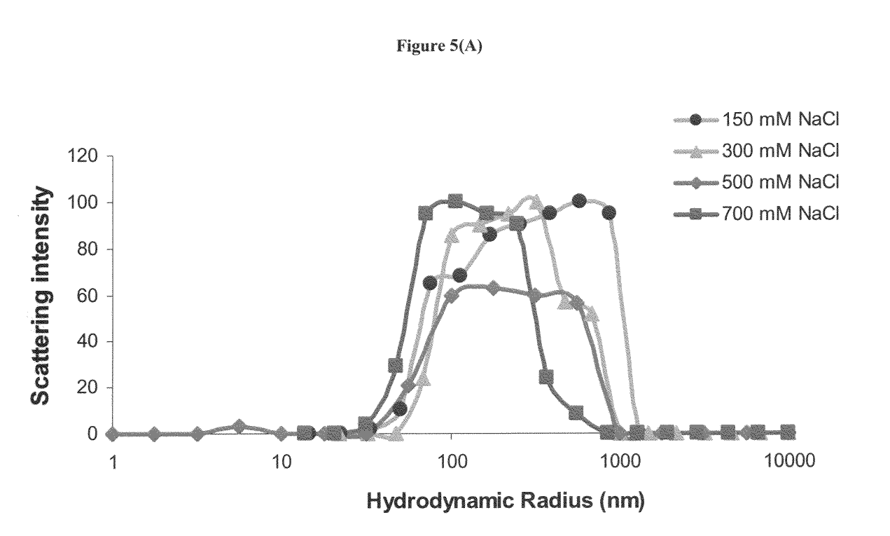

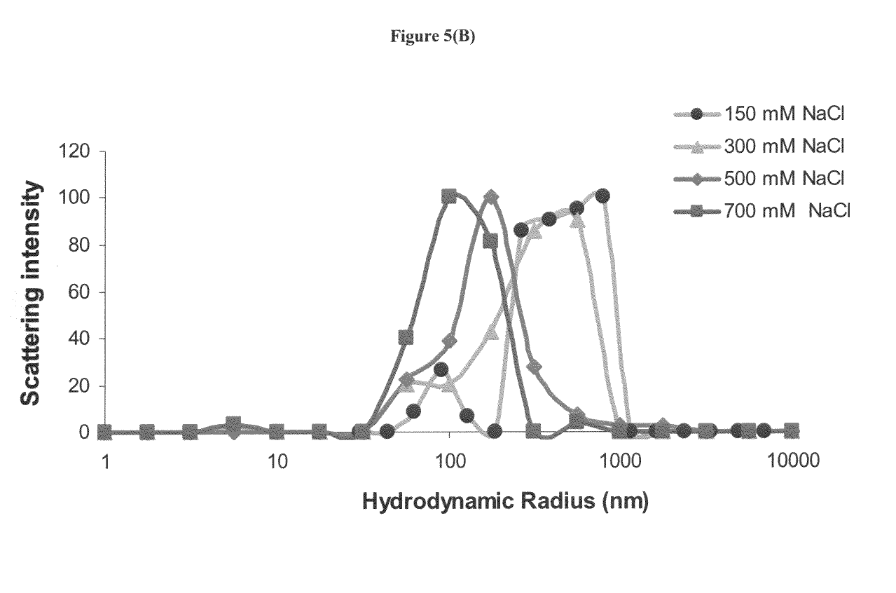

At 25° C., bPEI-HA/DNA complexes assembled in all of the examined salt solutions displayed a bimodal distribution. As the salt concentration increased, increasing numbers of complexes with smaller hydrodynamic radii appeared. After increasing the temperature to 37° C., most of the populations with smaller hydrodynamic radii shifted towards the populations with higher hydrodynamic radii. As can be seen from FIG. 5 and Table 2, the overall population of bPEI-HA/DNA complexes observed that were less than 100 nm in hydrodynamic radius was significantly more at 25° C. than at 37° C. across all the salt concentrations. Table 2 also shows that increasing the NaCl concentration caused the aggregates of bPEI-HA/DNA complexes to reduce in size.

TABLE 2
Rh values of bPEI-HA/DNA Complexes
in Varying Concentrations of NaCl

| NaCl Concentration | 150 mM | 300 mM | 500 mM | 700 mM |
|---|---|---|---|---|
| 25° C. | | | | |
| Distribution (nm) | 33.4-1000.0 | 47.3-1000.0 | 3.2-1000.0 | 2.9-847.0 |
| Mean Rh (nm) | 578 | 320 | 113 | 108 |
| 37° C. | | | | |
| Distribution (nm) | 187-1140 | 40-1000 | 30-1000 | 32-1000 |
| Mean Rh (nm) | 794 | 553 | 178 | 120 |

Since the distribution curves represented in FIG. 5 are subject to Rayleigh scattering, the intensity is not representative of the percentage population of the complexes at the corresponding hydrodynamic radius. However, the hydrodynamic radius distribution of the bPEI-HA/DNA complexes shifted to the left as salt concentration increased, implying that as more salt was added, the size of the aggregates was reduced. At 150 mM of NaCl concentration, maximum scattering was observed at 578 nm. As the salt concentration was increased to 700 mM, the maximum scattering shifted to 108 nm. Furthermore, the aggregate sizes show a bimodal distribution, and with increasing salt concentrations there is increase in the intensity of the lower mode and a simultaneous decrease in the intensity of the higher mode, indicating that the larger aggregates broke up into smaller fragments. The vector-DNA complexes were stable, as suggested by the electrophoresis gel in FIG. 4.

### Example 7

### Characterization of bPEI-HA/DNA Complexes Using Static Light Scattering Techniques

Static light scattering studies were used to address the question of whether an increase in temperature causes an increase in the size of aggregates of bPEI-HA/DNA complexes versus an increase in the hydrodynamic radius of individual complexes.

Stock solutions were prepared as described in Example 6. Using toluene as the reference solvent, scattering intensities were recorded for the samples and toluene in batch mode using a DAWN-EOS instrument (Wyatt Technology) equipped with a 30 mW GaAs laser at λ=690 nm for static light scattering. The first set of measurements was carried out at 25° C. after letting the solutions equilibrate for 5 minutes, and then the temperature of the cell was raised to 37° C. and samples were allowed to equilibrate to the higher temperature for 10-15 min. dn/dC ratios were calculated using toluene as the reference solvent on a Wyatt Technologies optilab device and were found to be 0.1 mg/mL. Each SLS measurement was repeated at least three times and one representative measurement was used to obtain the Zimm plot. The plot allows the determination of the weight-averaged molar mass (M_{w}) and the second virial coefficient (A₂) using the Zimm equation:

$$\frac{2 {{π}^{2} ( \tilde{n} ( dn / dC ) )}^{2}}{{λ}^{4} \times Na} \times \frac{C}{R ( ϑ )} = \frac{1}{{M}_{w}} + 2 {A}_{2} C$$

where ñ is the refractive index of the solvent, dn/dC is the refractive index of the polymer, λ, is the wavelength of the laser beam used, Na is Avogadro's number, C is the concentration of the polymer in solution (g/ml), R(θ) is the Reyleigh ratio, and M_{w} is the weight-average molecular weight (Da) to be derived.

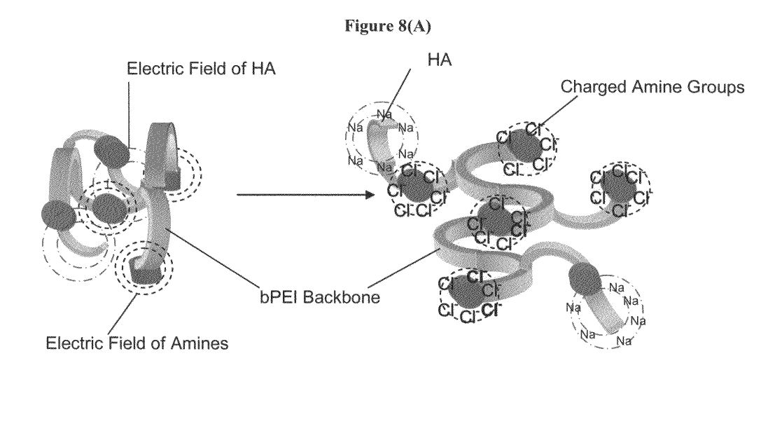

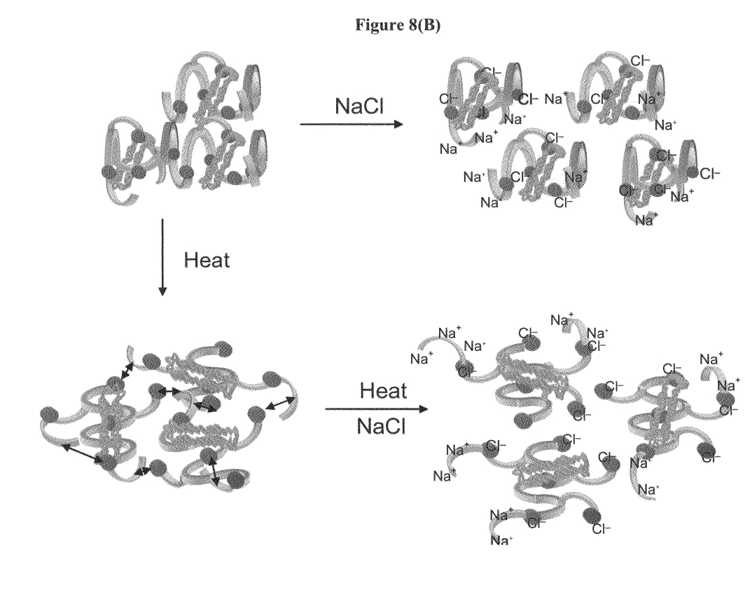

The scattering intensity of the polymer complexes at 25° C. and 37° C. was used to construct Zimm plots. The points for the Zimm plots were obtained by increasing dilutions of the sample with the respective salt solutions, and at eighteen different scattering angles. Using regression analysis, values of the points were fitted to a trend line which determined the A₂ as well as the molecular weight (not shown). When the molecular weight (derived from the Zimm equation) of the samples was plotted against the range of salt solutions, at 25° C. and 37° C., as represented in data in Table 3, representative data showed no difference between the molecular weight of the complexes. As shown in Table 3, the molecular weights of the complexes at any given salt concentration are comparable at 25° C. and 37° C. This result shows that the right-wards shift shown by the complexes with an increase in temperature was indeed due to the change in the hydrodynamic radius of these complexes. FIG. 8 provides a schematic summary of the influence of temperature and salt on bPEI-HA and bPEI-HA/DNA complexes.

TABLE 3
Molecular Weight of bPEI-HA/DNA Complexes

| NaCl concentration | 150 mM | 300 mM | 500 mM | 700 mM |
|---|---|---|---|---|
| Molecular Weight at | 2.02 × 10⁴ | 2.16 × 10⁴ | 8.41 × 10³ | 5.32 × 10³ |
| 25° C. | | | | |
| Molecular Weight at | 2.30 × 10⁴ | 2.09 × 10⁴ | 9.76 × 10³ | 4.44 × 10³ |
| 37° C. | | | | |

### Example 8

### Toxicity of bPEI-HA Conjugates and bPEI-HA/DNA Complexes Using a Live Dead Assay

hMSCs used for cell culture and cell based experiments were purchased from the laboratory of Dr. Darwin Prockop at the Tulane Center for Gene Therapy and grown using protocols established by Sekiya et al. Material for cell culture including α-Dulbecco's Modified Eagle's Medium (α-DMEM), glutamine, trypsin and PBS was obtained from Gibco (Carlsbad, Calif.). Plasmid DNA was purchased from Origene (Rockville, Md.).

A Live Dead assay was performed as follows: The toxicity of the synthesized bPEI-HA was compared to the toxicity of bPEI on hMSCs. hMSCs were seeded on 96 well, clear bottom plates at the density of 40,000 cells/cm². Cells were allowed to attach to the surface overnight after which they were exposed to bPEI, bPEI-HA, or their complexes with DNA (bPEI/DNA or bPEI-HA/DNA). The plasmid DNA used was pCMV-BMP2 (ClonTech, Mountainview, Calif.). The cells were exposed for 2 hours, 8 hours or 24 hours after which the cells were washed with PBS and complete media (α-MEM, 20% FBS, 10% glycine, 10% penicilline-streptomycine) was added to the wells. The cells were tested for viability at 2 hours, 8 hours, 24 hours, 48 hours, 72 hours and 96 hours using the Live/Dead Viability/Cytotoxicity reagent (Molecular Probes) (4 μM Ethidium homodimer-1 (EthD-1) and 2 μM of Calcein-AM) as per manufacturer's instructions. The cells were washed with PBS prior to addition of 100 μl Live/Dead reagent. The plates were incubated for 30 minutes. hMSCs untreated with any chemicals and grown in complete medium were used as live control cells and cells treated with 70% methanol for 30 minutes were used as the dead control. Fluorescence was measured using a fluorescent microplate reader (FLx800 Bio-TEK instruments) equipped with 485/582 (excitation/emission) filters sets to measure calcein (green fluorescence) and 528/620 for EthD-1 (red fluorescence). The fraction of live and dead cells was calculated as described by Temenoff et al. (2003). Furthermore, the live and dead cells were visualized by fluorescence microscopy using Nikon-Eclipse E600 and software Image-Pro Plus 5.1.

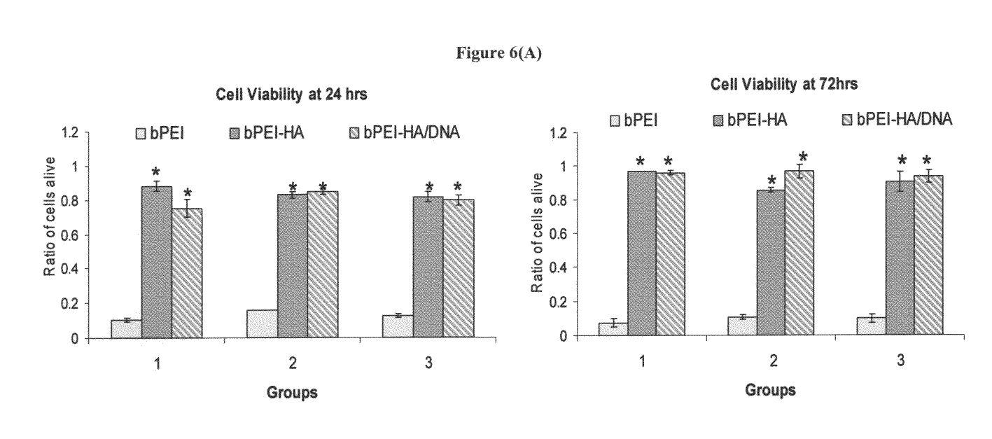

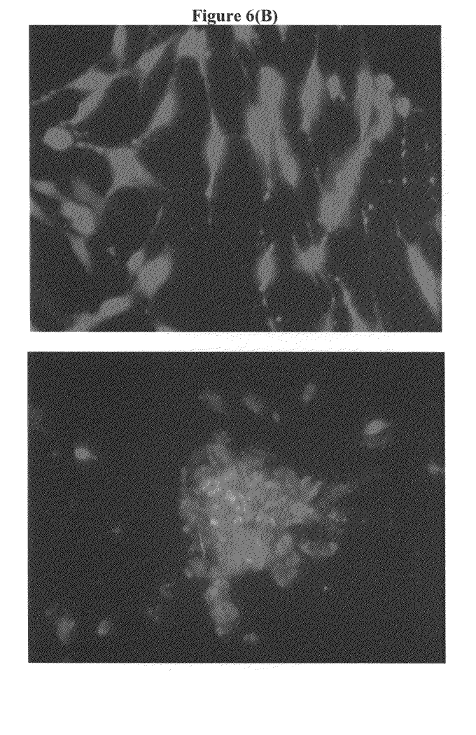

As shown in FIG. 6(A), across all time points, bPEI was toxic to hMSCs. Less than 10% of hMSCs survived with exposed to bPEI alone. However, bPEI-HA exhibited reduced toxicity to hMSCs, with more than 95% cells surviving at every time point. The cells survived just as well when bPEI-HA was complexed with different concentrations of DNA. Statistical analysis was performed between groups for the live-dead assay. Groups were analyzed with ANOVA with a p-value <0.05 and pairwise comparison was performed using the Tukey test.

These data were corroborated both quantitatively as well as visually with fluorescent microscopy. All cells treated either with bPEI-HA conjugates or bPEI-HA/DNA complexes appeared healthy, showing the normal morphology of hMSCs. Visually, both bPEI as well as bPEI/DNA complexes showed the presence of clusters of cells, as well as reduced cell density on the surfaces of the cells, representing apoptotic cells (FIG. 6(B)).

### Example 9

### Efficiency of Transfection Based on Temperature and Salt Concentration at the time of bPEI-HA/DNA Complex Assembly

hMSCs were purchased from the laboratory of Dr. Darwin Prockop at Tulane Center for Gene Therapy. hMSCs were plated on 6 well plates at the density of 5×103 cells/cm² (˜50,000 cells/well). hMSCs were allowed to attach overnight in the presence of complete medium after which the medium was replaced by DMEM. Before the cells were transfected with the plasmids of interest, the cells cycles were synchronized with the assumption that the doubling time of hMSCs is approximately 30 hours. (Colter et al. 2000) The cells were incubated in FBS free medium (DMEM) for 30 hours which limited the amount of nutrients provided to the cells. After 30 hours, the cells were treated with complete medium for 6 hours to allow reactivation of the cells through the cell cycle.

bPEI-HA/DNA complexes were assembled at C:A ratios of 7.5:1 at 150 mM of NaCl at 25° C. and 37° C. and at 500 mM of NaCl at 25° C. and 37° C. The complexes were centrifuged as described before and allowed to stand for 1 hr to allow complete assembly. The cells were treated with the bPEI-HA/DNA complexes as described above with two sets of cells acting as control: cells treated with DMEM only, and cells treated with naked DNA in the presence of DMEM. Flow cytometry, as described in Example 11, was used to determine the percentage of cells transfected.

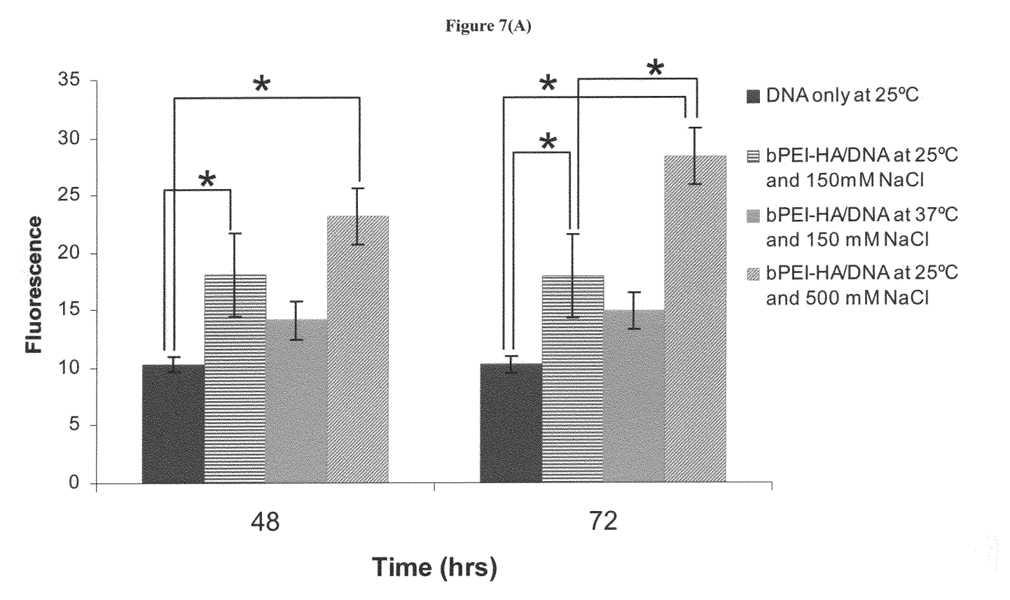

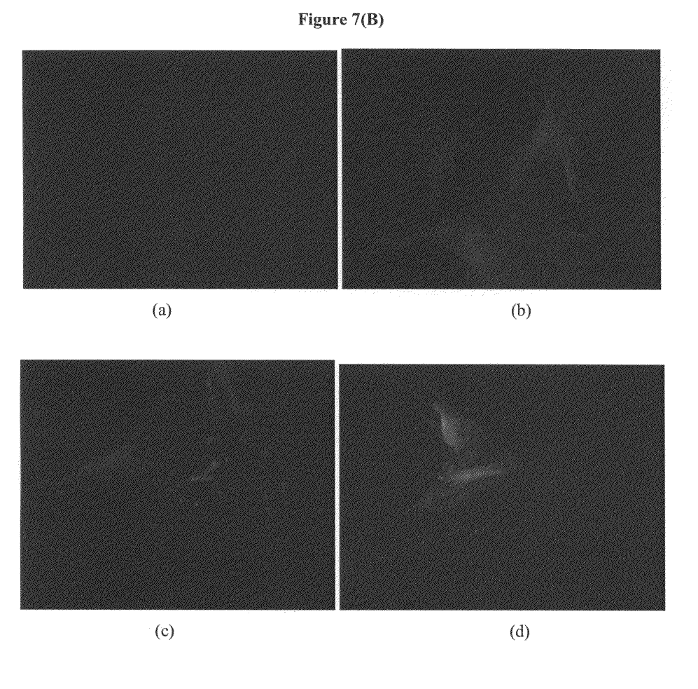

The bPEI-HA/DNA complexes assembled in 500 mM NaCl were transfected into hMSCs over samples assembled in 150 mM NaCl. Mortality associated with the 500 mM samples was comparable to that of the 150 mM samples (<5%). As can be seen from FIG. 7(A), samples treated with bPEI-HA/DNA complexes assembled in 150 mM of NaCl had statistical improvement in transfection efficiency as compared to DNA alone. Samples that were assembled at 37° C. had lower transfection efficiency than the samples assembled at 25° C., however this difference was not statistically significant. However, when complexes were assembled at 25° C. in 500 mM of NaCl, the transfection efficiency was increased at least two-fold over DNA alone. This efficiency was statistically better than the transfection efficiency of complexes assembled in 150 mM NaCl. Transfected cells expressing green fluorescent protein were also observed under fluorescence microscopy as shown in FIG. 8(B).

### Example 10

### Efficiency of Transfection Based on C:A Ratios

bPEI-HA/DNA complexes were assembled as described above at the following C:A ratios: 5.5:1, 6.5:1, 7.5:1, 8.5:1, 10.5:1, and 13.5:1 at 500 mM NaCl. The complexes were assembled at 25° C. and were allowed to stand for 1 hour after centrifugation to allow complete assembly. hMSCs were washed with PBS, and the bPEI-HA/DNA complexes were added to the wells in the presence of 200 μl of DMEM. Additionally, one set of hMSCs receiving only DMEM and another set receiving naked DNA in DMEM were used as controls. The cells were placed in a 37° C. incubator for 24 hours after which the medium was supplemented with complete medium. The cells were further incubated for either 48 hours or 72 hours after which the cells were tested for fluorescence using a flow cytometer.

All the complexes transfected significantly better than the negative controls, however there was no statistical difference between the transfection efficiencies of the above complexes. Ratios of 5.5:1, 6.5:1 and 7.5:1 had slightly higher transfection than the rest of the complexes; however, the difference was not statistically different.

### Example 11

### Flow Cytometry

The following procedures were utilized in the above examples whenever flow cytometry techniques were called for.

To fix the cells, the cells were washed three times with sterile PBS to remove any dead cells as well as any residual gene delivery agents. Cell-wells were treated with 0.5 ml of 0.5× trypsin (Gibco, Bethesda, Md.) and were placed in the incubator for 3 minutes to allow the cells to detach from the wells. Cells were then treated with complete medium to stop the reaction of trypsin. The cells were removed from the cell wells and placed in Falcon tubes. The cells were centrifuged for 10 min at 10000 rpm to separate the medium from the cells. Following centrifugation the medium was aspirated and replaced with 1% formaldehyde solution for 1 hour in an ice bucket. The cells were centrifuged again and the sterile PBS replaced the 1% formaldehyde solution. The cells were suspended in PBS with repeated pipetting.

The cells were counted using flow cytometry (Becton Dickenson FACS Scan) at high flow and Cell Quest Pro software. To identify the location of the cells on the graph, sterile PBS was run through the flow cytometer to identify the background signal. A simple suspension of cells in PBS was then run through the FACS machine to identify the location of the population of cells. The FACS machine was further calibrated to register green fluorescence emitted by cells that were successfully transfected, while a separate channel recorded the total number of cells passing through the capillary of the FACS. 5000 events were counted for each sample.

Statistical analysis was performed between groups for the flow cytometry transfection studies. Groups were analyzed with ANOVA with a p-value <0.05 and pairwise comparison was performed using the Tukey test.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

Andrey V. Dobrynin, R. H. C. M. R., Polyampholytes. Journal of Polymer Science Part B: Polymer Physics 2004, 42, (19), 3513-3538.
Caplan, A. I.; Bruder, S. P., Mesenchymal stem cells: building blocks for molecular medicine in the 21st century. Trends Mol Med 2001, 7, (6), 259-64.
Chow, G.; Knudson, C. B.; Knudson, W., Expression and cellular localization of human hyaluronidase-2 in articular chondrocytes and cultured cell lines. Osteoarthritis and Cartilage 2006, 14, (9), 849.
Colter, D. C.; Class, R.; DiGirolamo, C. M.; Prockop, D. J., Rapid expansion of recycling stem cells in cultures of plastic-adherent cells from human bone marrow. PNAS 2000, 97, (7), 3213-3218.
Fischer, D.; Li, Y.; Ahlemeyer, B.; Krieglstein, J.; Kissel, T., In vitro cytotoxicity testing of polycations: influence of polymer structure on cell viability and hemolysis. Biomaterials 2003, 24, (7), 1121.
Gao, H.; Shi, W.; Freund, L. B., From The Cover: Mechanics of receptor-mediated endocytosis. PNAS 2005, 102, (27), 9469-9474.
Godbey, W. T.; Wu, K. K.; Mikos, A. G., Poly(ethylenimine) and its role in gene delivery. Journal of Controlled Release 1999, 60, (2-3), 149.
Godbey, W. T.; Wu, K. K.; Mikos, A. G., Size matters: molecular weight affects the efficiency of poly(ethylenimine) as a gene delivery vehicle. J Biomed Mater Res 1999, 45, (3), 268-75.
Grosse, S.; Aron, Y.; Honore, I.; Thevenot, G.; Danel, C.; Roche, A. C.; Monsigny, M.; Fajac, I., Lactosylated polyethylenimine for gene transfer into airway epithelial cells: role of the sugar moiety in cell delivery and intracellular trafficking of the complexes. J Gene Med 2004, 6, (3), 345-56.
Hua, Q.; Knudson, C. B.; Knudson, W., Internalization of hyaluronan by chondrocytes occurs via receptor-mediated endocytosis. J Cell Sci 1993, 106 (Pt 1), 365-75.
K. A. Narh, A. K., Precipitation effects in polyelectrolytes on addition of salts. Journal of Polymer Science Part B: Polymer Physics 1993, 31, (2), 231-234.
Kassem, M., Stem Cells. Potential Therapy for Age-Related Diseases. Annals of the New York Academy of Sciences 2006, 1067, (1), 436-442.
Knudson, C. B., Hyaluronan and CD44: strategic players for cell-matrix interactions during chondrogenesis and matrix assembly. Birth Defects Res C Embryo Today 2003, 69, (2), 174-96.
Knudson, C. B.; Knudson, W., Hyaluronan and CD44: modulators of chondrocyte metabolism. Clin Orthop Relat Res 2004, (427 Suppl), S152-62.
Knudson, W.; Casey, B.; Nishida, Y.; Eger, W.; Kuettner, K. E.; Knudson, C. B., Hyaluronan oligosaccharides perturb cartilage matrix homeostasis and induce chondrocytic chondrolysis. Arthritis Rheum 2000, 43, (5), 1165-74.
Kvam, B. J.; Atzori, M.; Toffanin, R.; Paoletti, S.; Biviano, F., Carbohydr. Res. 1992, 230, 1-13.
Lisignoli, G.; Cristino, S.; Piacentini, A.; Toneguzzi, S.; Grassi, F.; Cavallo, C.; Zini, N.; Solimando, L.; Mario Maraldi, N.; Facchini, A., Cellular and molecular events during chondrogenesis of human mesenchymal stromal cells grown in a three-dimensional hyaluronan based scaffold. Biomaterials 2005, 26, (28), 5677.
Longobardi, L.; O'Rear, L.; Aakula, S.; Johnstone, B.; Shimer, K.; Chytil, A.; Horton, W. A.; Moses, H. L.; Spagnoli, A., Journal of Bone and Mineral Research 2006, 21, (4), 626-636.
Meinel, L.; Hofmann, S.; Betz, O.; Fajardo, R.; Merkle, H. P.; Langer, R.; Evans, C. H.; Vunjak-Novakovic, G.; Kaplan, D. L., Osteogenesis by human mesenchymal stem cells cultured on silk biomaterials: Comparison of adenovirus mediated gene transfer and protein delivery of BMP-2. Biomaterials 2006, 27, (28), 4993.
Ohno, S.; Im, H. J.; Knudson, C. B.; Knudson, W., Hyaluronan oligosaccharide-induced activation of transcription factors in bovine articular chondrocytes. Arthritis Rheum 2005, 52, (3), 800-9.
Peterson, R. S.; Andhare, R. A.; Rousche, K. T.; Knudson, W.; Wang, W.; Grossfield, J. B.; Thomas, R. O.; Hollingsworth, R. E.; Knudson, C. B., CD44 modulates Smad1 activation in the BMP-7 signaling pathway. J. Cell Biol. 2004, 166, (7), 1081-1091.
Rensen, P. C. N.; Sliedregt, L. A. J. M.; Ferns, M.; Kieviet, E.; van Rossenberg, S. M. W.; van Leeuwen, S. H.; van Berkel, T. J. C.; Biessen, E. A. L., Determination of the Upper Size Limit for Uptake and Processing of Ligands by the Asialoglycoprotein Receptor on Hepatocytes in Vitro and in Vivo. J. Biol. Chem. 2001, 276, (40), 37577-37584.
Sekiya, I.; Larson, B. L.; Smith, J. R.; Pochampally, R.; Cui, J.-G.; Prockop, D. J., Expansion of Human Adult Stem Cells from Bone Marrow Stroma: Conditions that Maximize the Yields of Early Progenitors and Evaluate Their Quality. Stem Cells 2002, 20, (6), 530-541.
Seyfried, N. T.; McVey, G. F.; Almond, A.; Mahoney, D. J.; Dudhia, J.; Day, A. J., Expression and Purification of Functionally Active Hyaluronan-binding Domains from Human Cartilage Link Protein, Aggrecan and Versican: FORMATION OF TERNARY COMPLEXES WITH DEFINED HYALURONAN OLIGOSACCHARIDES. J. Biol. Chem. 2005, 280, (7), 5435-5448.
Simmons, C. A. C. A.; Matlis, S.; Thornton, A. J. A. J.; Chen, S.; Wang, C. Y. C.-Y.; Mooney, D. J. D. J., Cyclic strain enhances matrix mineralization by adult human mesenchymal stem cells via the extracellular signal-regulated kinase (ERK1/2) signaling pathway. Journal of Biomechanics 2003, 36, (8), 1087.
Solchaga, L. A.; Temenoff, J. S.; Gao, J.; Mikos, A. G.; Caplan, A. I.; Goldberg, V. M., Repair of osteochondral defects with hyaluronan- and polyester-based scaffolds. Osteoarthritis Cartilage 2005, 13, (4), 297-309.
Temenoff, J. S.; Shin, H.; Conway, D. E.; Engel, P. S.; Mikos, A. G., In Vitro Cytotoxicity of Redox Radical Initiators for Cross-Linking of Oligo(poly(ethylene glycol) fumarate) Macromers. Biomacromolecules 2003, 4, (6), 1605-1613.
Thomas, M.; Klibanov, A. M., Enhancing polyethylenimine's delivery of plasmid DNA into mammalian cells. Proc Natl Acad Sci USA 2002, 99, (23), 14640-5.
Toole, B. P., Hyaluronan in morphogenesis. Journal of Internal Medicine 1997, 242, (1), 35-40.
Toole, B. P., Hyaluronan in morphogenesis. Seminars in Cell & Developmental Biology 2001, 12, (2), 79.
Toole, B. P.; Jackson, G.; Gross, J., Hyaluronate in morphogenesis: inhibition of chondrogenesis in vitro. Proc Natl Acad Sci USA 1972, 69, (6), 1384-6.
Underhill, C., CD44: the hyaluronan receptor. J Cell Sci 1992, 103, (2), 293-298.
Vikas K. Sharma, M. T. A. M. K., Mechanistic studies on aggregation of polyethylenimine-DNA complexes and its prevention. Biotechnology and Bioengineering 2005, 90, (5), 614-620.
Volpi, N.; Maccari, F., Biochimie. 2003, 85, 619-25.
Wagner, E.; Cotten, M.; Foisner, R.; Birnstiel, M. L., Transferrin-Polycation-DNA Complexes: The Effect of Polycations on the Structure of the Complex and DNA Delivery to Cells. PNAS 1991, 88, (10), 4255-4259.
Zanta, M. A.; Boussif, O.; Adib, A.; Behr, J. P., In vitro gene delivery to hepatocytes with galactosylated polyethylenimine. Bioconjug Chem 1997, 8, (6), 839-44.

What is claimed is:

1. A composition comprising a plurality of hyaluronic acid hexamers covalently attached to a branched polyethylenimine.

2. The composition of claim 1 further comprising at least one DNA molecule.

3. The composition of claim 1 further comprising a portion of at least one DNA molecule.

4. The composition of claim 1 wherein the plurality of hyaluronic acid hexamers are covalently attached to at least one side chain of the branched polyethylenimine.

5. A method comprising:
providing a plurality of hyaluronic acid hexamers and a branched polyethylenimine; and
allowing a hexamer of hyaluronic acid to covalently attach to a branched polyethylenimine to form a conjugate.

6. The method of claim 5 further comprising the step of complexing the conjugate with at least one DNA molecule.

7. The method of claim 6 wherein the step of complexing the conjugate with at least one DNA molecule comprises:
placing the conjugate into an aqueous salt solution;
placing the at least one DNA molecule into the aqueous salt solution; and
allowing the conjugate and DNA molecule to form a complex.

8. The method of claim 7 wherein the aqueous salt solution comprises a salt concentration of at least 150 mM.

9. The method of claim 7 wherein the aqueous salt solution comprises sodium chloride.

10. The method of claim 5 wherein the plurality of hyaluronic acid hexamers are covalently attached to at least one side chain of the branched polyethylenimine.

11. The method of claim 5 wherein the step of allowing a hexamer of hyaluronic acid to covalently attach to a branched polyethylenimine to form a conjugate comprises a reductive amination reaction.

12. The method of claim 5 further comprising the step of purifying the conjugate.

13. The method of claim 5 further comprising the step of lyophilizing the conjugate.

14. A method comprising:
providing a conjugate comprising a plurality of hyaluronic acid hexamers covalently attached to a branched polyethylenimine; and
administering the conjugate to a cell.

15. The method of claim 14 wherein the cell is a mesenchymal stem cell.

16. The method of claim 14 wherein the step of administering the conjugate to the cell comprises allowing the conjugate to interact with a CD44 receptor on the cell.

17. The method of claim 14 wherein the conjugate further comprises at least one DNA molecule.

18. The method of claim 17 wherein the at least one DNA molecule is complexed with the conjugate by the steps of:
placing the conjugate into an aqueous salt solution;
placing the at least one DNA molecule into the aqueous salt solution; and
allowing the conjugate and DNA molecule to form a complex.

19. The method of claim 18 wherein the aqueous salt solution comprises a salt concentration of at least 150 mM.

20. The method of claim 18 wherein the at least one DNA molecule is complexed with the conjugate at a temperature below about 37° C.
